(19) United States
(12) Patent Application Publication
(10) Pub. No.: US 2023/0381474 A1
(43) Pub. Date: Nov. 30, 2023

(54) Burn Treatment Glove

(71) Applicant: Eddie Holloway, Chicago, IL (US)

(72) Inventor: Eddie Holloway, Chicago, IL (US)

(21) Appl. No.: 17/752,972

(22) Filed: May 25, 2022

(51) Int. Cl. A61M 35/00; A41D 19/00; A41D 31/10; A41D 31/18; A61K 31/167

(52) U.S. Cl. CPC A61M 35/10; A41D 19/0006; A41D 31/10; A41D 31/185; A61K 31/167; A61M 2205/0216; A61M 2205/0205

(57) ABSTRACT

A burn treatment glove for alleviating pain resulting from a burn and preventing infection includes a glove comprising a contoured layer constructed of an elastically stretchable, liquid-impermeable material. An elastic tension force in the glove compresses a hand wearing the glove for improved blood flow and reduced swelling. A medicinal liquid coats a portion of the interior surface, which applies to a burn on the skin of the hand when the burn contacts the liquid.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medicated gloves and more particularly pertains to a new medicated glove for alleviating pain resulting from a burn and preventing infection.

### (2) Description of Related Art Including Information Disclosed Under 37 CFR 1.97 and 1.98

The prior art relates to medicated gloves which apply a medicated topical treatment to a hand of a user through various means, including a lotion absorbent material lining a lotion impervious material and coating an inner surface with a dehydrated material to be hydrated by the user's perspiration. But the prior art does not disclose a medicated glove that simultaneously applies a medication to the skin and utilizes a compression treatment for reducing painful swelling and increased blood flow.

### BRIEF SUMMARY OF THE INVENTION

An embodiment of the disclosure meets the needs presented above by generally comprising a glove comprising a contoured layer constructed of an elastically stretchable, liquid-impermeable material. An elastic tension force in the glove compresses a hand wearing the glove for improved blood flow and reduced swelling. A medicinal liquid coats a portion of the interior surface, which applies to a burn on the skin of the hand when the burn contacts the liquid.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a burn treatment glove according to an embodiment of the disclosure.

FIG. 2 is a cross-sectional front view of an embodiment of the disclosure.

FIG. 3 is an in-use front view of an embodiment of the disclosure.

FIG. 4 is an in-use exploded view of an embodiment of the disclosure.

### DETAILED DESCRIPTION OF THE INVENTION

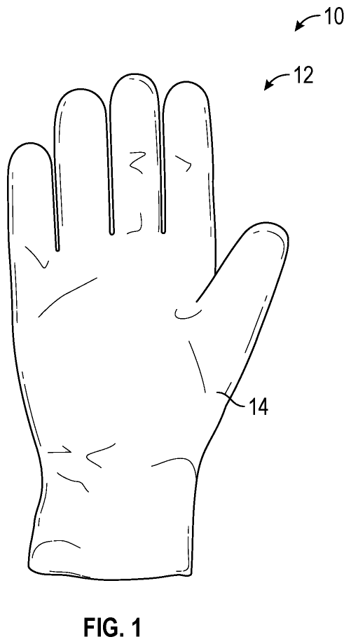

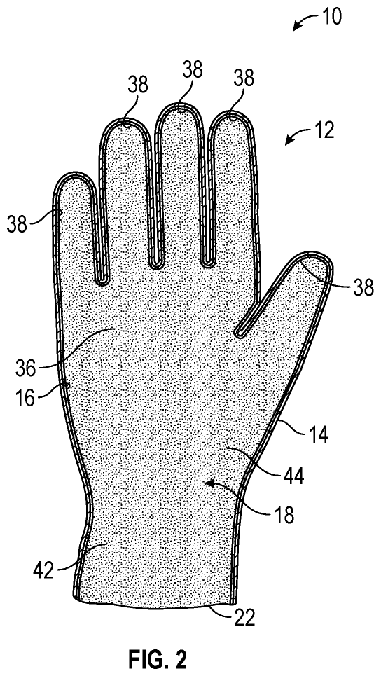

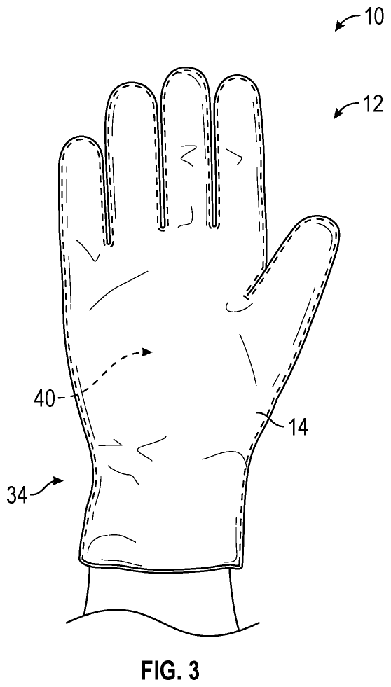

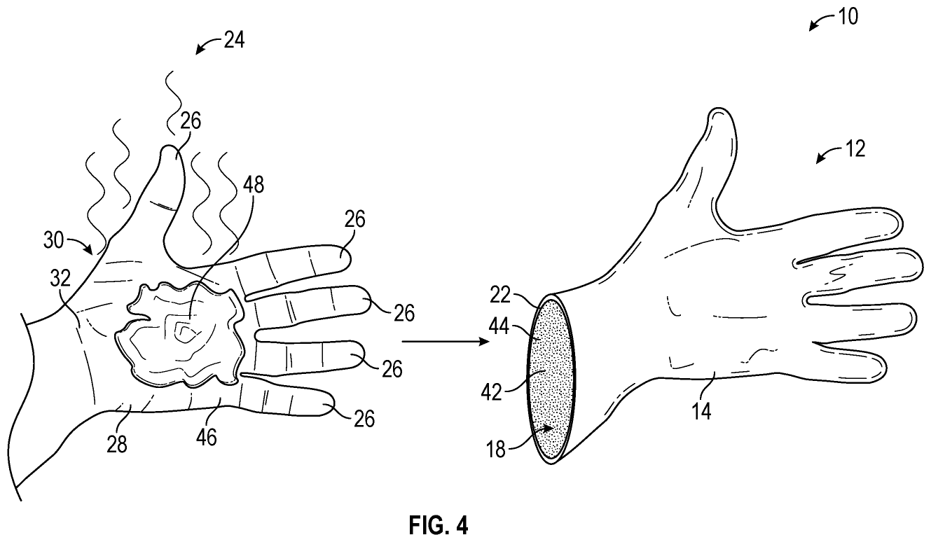

With reference now to the drawings, and in particular to FIGS. 1 through 4 thereof, a new medicated glove embodying the principles and concepts of an embodiment of the disclosure and generally designated by the reference numeral 10 will be described.

As best illustrated in FIGS. 1 through 6, the burn treatment glove 10 generally comprises a glove 12 comprising a contoured layer 14 constructed of a liquid-impermeable, elastically stretchable material, such as latex, nitrile, or the like. The glove 12 has an interior surface 16 defining a chamber 18 and is configured for elastically stretching to receive a hand 24 through an opening 22 of the chamber 18. The interior surface 16 defines a plurality of sub-chambers 20, wherein each sub-chamber 20 is in environmental communication with the chamber 18. Each sub-chamber 20 is configured for elastically stretching to receive an associated digit 26 of a plurality of digits 26 of the hand 24. The glove 12 is in a worn position 34 when each digit 26 is positioned within an associated sub-chamber 20 of the plurality of sub-chambers 20 and a palm 28 of the hand 24, a back 30 of the hand 24, and a wrist 32 of the hand 24 are positioned within the chamber 18. The glove 12 undergoes an elastic tension force when stretched around the hand 24 and is configured to compress the hand 24 when the glove 12 is in the worn position 34.

The interior surface 16 includes a plurality of portions, the plurality of portions including a palm portion 36, a back portion 40, a wrist portion 42, and a plurality of digit portions 38, wherein the palm portion 36 covers the palm 28, the back portion 40 covers the back 30 of the hand 24, the wrist portion 42 covers the wrist 32, and each digit portion 38 of the plurality of digit portions 38 covers an associated digit 26 when the glove 12 is in the worn position 34. A liquid 44 including lidocaine and an antibiotic coats each portion of the interior surface 16. Lidocaine is a local anesthetic commonly used to soothe burns. An antibiotic is an antibacterial agents used for treating and preventing bacterial infections. The liquid 44 is configured to apply to a skin 46 of the hand 24 when the skin 46 contacts the liquid 44. The liquid 44 is configured to apply to a burn 48 on the skin 46 of the hand 24 when the burn 48 contacts the liquid.

In use, a hand 24 including a burn 48 is positioned inside the glove 12. The glove 12 compresses the hand 24, promoting blood flow and counteracting painful swelling. The burn 48 contacts the liquid 44 including the lidocaine and the antibiotic. The lidocaine soothes the pain, and the antibiotic prevents bacteria from infecting the burn 48.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A burn treatment glove comprising:
a glove, said glove comprising a contoured layer constructed of a liquid-impermeable material, said material being elastically stretchable, said glove having an interior surface defining a chamber, said glove being configured for elastically stretching to receive a hand through an opening of said chamber, wherein said glove undergoes an elastic tension force when stretched around said hand, said glove being configured to compress the hand when the hand is in said chamber;
a liquid coating a portion of said interior surface, said liquid being a medicinal agent, said liquid being configured to apply to a skin of the hand when the skin contacts said liquid, said liquid being configured to apply to a burn on the skin of the hand when the burn contacts said liquid.

2. The device of claim 1, wherein;
said interior surface defines a plurality of sub-chambers, each said sub-chamber being in environmental communication with said chamber, each said sub-chamber being configured for elastically stretching to receive an associated digit of a plurality of digits of the hand,
said glove is in a worn position when the glove is configured to receive each digit within an associated sub-chamber of said plurality of sub-chambers and a palm of the hand, a back of the hand, and a wrist of the hand are positioned within said chamber,
said glove undergoes an elastic tension force when stretched around said hand, said glove being configured to compress the hand when said glove is in said worn position.

3. The device of claim 2, wherein said interior surface includes a plurality of digit portions, each digit portion of said plurality of digit portions covering an associated digit when said glove is in said worn position, wherein said liquid coats a digit portion.

4. The device of claim 2, wherein said interior surface includes a palm portion, said palm portion covering the palm of the hand when said glove is in said worn position, wherein said liquid coats said palm portion.

5. The device of claim 2, wherein said interior surface includes a back portion, said palm portion covering the back of the hand when said glove is in said worn position, wherein said liquid coats said back portion.

6. The device of claim 2, wherein said interior surface includes a wrist portion, said palm portion covering the wrist of the hand when said glove is in said worn position, wherein said liquid coats said wrist portion.

7. The device of claim 1, wherein said liquid includes lidocaine.

8. The device of claim 1, wherein said liquid includes an antibiotic.

9. The device of claim 1, wherein said glove is constructed of latex.

10. The device of claim 1, wherein said glove is constructed of nitrile.

11. A burn treatment glove comprising:
a glove, said glove comprising a contoured layer constructed of a liquid-impermeable material, said material being elastically stretchable, said glove having an interior surface defining a chamber, said glove being configured for elastically stretching to receive a hand through an opening of said chamber,
wherein said interior surface defines a plurality of sub-chambers, each said sub-chamber being in environmental communication with said chamber, each said sub-chamber being configured for elastically stretching to receive an associated digit of a plurality of digits of the hand,
wherein said glove is in a worn position when the glove is configured to receive each digit within an associated sub-chamber of said plurality of sub-chambers and a palm of the hand, a back of the hand, and a wrist of the hand are positioned within said chamber,
wherein said glove undergoes an elastic tension force when stretched around said hand, said glove being configured to compress the hand when said glove is in said worn position;
wherein said interior surface includes a plurality of portions, said plurality of portions including a palm portion, a back portion, a wrist portion, and a plurality of digit portions, wherein said palm portion covers the palm, said back portion covers the back of the hand, said wrist portion covers the wrist, and each digit portion of said plurality of digit portions covers an associated digit when said glove is in said worn position; and
a liquid coating each said portion of said interior surface, said liquid including lidocaine and an antibiotic, said liquid being configured to apply to a skin of the hand when the skin contacts said liquid, said liquid being configured to apply to a burn on the skin of the hand when the burn contacts said liquid.
